(19) United States
(12) Patent Application Publication
(10) Pub. No.: US 2010/0068708 A1
(43) Pub. Date: Mar. 18, 2010

(54) Methods for Identifying Compounds that Modulate WNT Signaling in Cancer Cells

(75) Inventors: John Hood, San Diego, CA (US); Charlene F. Barroga, San Diego, CA (US); Dennis Carson, La Jolla, CA (US); Desheng Lu, San Diego, CA (US)

Correspondence Address: FISH & RICHARDSON P.C., PO BOX 1022, MINNEAPOLIS, MN, 55440-1022, US

(73) Assignee: Wintherix LLC, San Diego, CA (US)

(21) Appl. No.: 12/437,318

(22) Filed: May 7, 2009

Related U.S. Application Data
(60) Provisional application No. 61/051,322, filed on May 7, 2008.
(60) Provisional application No. 61/083,870, filed on Jul. 25, 2008.

(51) Int. Cl. C12Q 1/68; C07D 473/32

(52) U.S. Cl. 435/6; 544/276

(57) ABSTRACT

Provided herein are methods for screening compounds for their ability to modulate Wnt signaling in cancer cells.

### CROSS-REFERENCE

This application claims the benefit of U.S. Provisional Application No. 61/051,322, filed May 7, 2008, and U.S. Provisional Application No. 61/083,870 filed Jul. 25, 2008, which applications is incorporated herein by reference.

The invention relates to assays for screening compounds for their affects on cells having an active Wnt signaling pathway.

### BACKGROUND OF THE INVENTION

Wnt signaling affects fundamental development pathways by regulating cell proliferation and differentiation, and is active in many cancers, including colon cancer, leukemias, breast cancer, hepatocellular carcinoma, prostate cancer, and melanoma.

In the canonical Wnt pathway, binding of Wnt, a secreted glycoprotein, to the Frizzled receptor leads to accumulation of beta-catenin in the cytoplasm, resulting in its translocation to the nucleus where it binds to the HMG binding proteins of the LEF/TCF family to activate transcription of Wnt target genes. In the absence of Wnt signaling, beta-catenin is continuously degraded by the ubiquitin pathway; the turnover of beta-catenin is mediated by the beta-catenin destruction complex, which includes the proteins APC, GSK3-beta, and axin. GSK3-beta phosphorylates beta-catenin, marking it for destruction. During Wnt signaling, the beta-catenin destruction complex is disrupted, such that beta catenin phosphorylation is prevented, so that beta-catenin accumulates and then enters the nucleus, where it binds to members of the LEF/TCF family of HMG DNA binding proteins.

While the LEF/TCF family members LEF-1, TCF-1, and TCF-4 do not themselves activate transcription, they do have the ability to bind and bend DNA via their HMG domains. In at least some cases, LEF/TCF proteins bind DNA and recruit transcriptional repressors in the absence of beta-catenin. During Wnt signaling, when beta-catenin becomes available in the nucleus, the repressors are displaced by beta-catenin, which mediates interactions with transcriptional activators. Gene targets of the Wnt pathway include c-Myc, Cyclin D1, Cdx, MMP7, c-Myb, c-Kit, PPARsigma, Axin2, Sp5, DKK4, Bcl-X, LEF-1 itself, and others.

LEF-1, TCF-1, and TCF-4 are alternatively spliced genes. Splice variants of these DNA binding proteins lead to variants having different domains in their C-terminal tails (J. Cell Sci 120: 385-393 (2007)). In addition, both LEF-1 and TCF-1 have two promoters: each has a first promoter that directs expression of a transcript encoding a full length protein and a second promoter within a downstream intron of the gene that directs expression of an N-terminally truncated version. The N-terminally truncated versions of LEF-1 and TCF-1 (deltaN-LEF-1 and deltaN-TCF-1) lack the beta-catenin binding domain of these proteins but retain their DNA binding domains, allowing these isoforms of LEF-1 and TCF-1 to act as dominant negatives and downregulate the canonical Wnt signaling pathway.

### SUMMARY OF THE INVENTION

Provided herein are methods for screening compounds for their ability to modulate Wnt signaling in cancer cells.

In one aspect, provided herein are methods for identifying a compound that modulates Wnt signaling in a cancerous cell, in which the method includes: providing a cancerous cell that comprises a reporter gene regulated by a promoter modulated by the interaction between TCF/LEF and β-catenin, providing a noncancerous cell that comprises the reporter gene regulated by a promoter modulated by the interaction between TCF/LEF and β-catenin, contacting the cancerous cell and the noncancerous cell with a test compound, detecting a signal from the reporter gene in the cancerous cell contacted with the test compound and a signal from the reporter gene in the cancerous cell not contacted with the test compound, and detecting a signal from the reporter gene in the noncancerous cell contacted with the test compound and a signal from the reporter gene in the noncancerous cell not contacted with the test compound. The method further includes identifying a test compound that modulates the signal from expression of the reporter gene in the cancerous cell, but does not modulate the signal from expression of the reporter gene in the non-cancerous cell.

A cancerous cell used in the methods can be any cancerous cell, and can be, as nonlimiting examples, a colon cancer cell, a leukemia cell, a lymphoma cell, a melanoma cell, a breast cancer cell, a prostate cancer cell, a hepatocarcinoma cell, a lung cancer cell, an ovarian cancer cell, a uterine cancer cell, a cervical cancer cell, or a head-and-neck cancer cell.

A noncancerous cell used in the methods can be any noncancerous cell, and can be, as nonlimiting examples, a HEK293 cell, a COS-7 cell, a CHO cell, a NIH/3T3 cell, or a noncancerous colon cell, epithelial cell, skin cell, B cell, pre-B cell, T cell, pre-T cell, breast cell, prostate cell, liver cell, lung cell, ovarian cell, uterine cell, or cervical cell.

Promoters modulated by the interaction between TCF/LEF and β-catenin are any promoters that are upregulated, downregulated, repressed, or activated by a TCF/LEF protein bound to or acted on by β-catenin, including synthetic promoters, naturally-occurring promoters, portions of naturally-occurring promoters, variants of naturally-occurring promoters, chimeric promoters, etc.

Also provided is a method of identifying a compound that modulates Wnt signaling, in which the method includes: providing a cell that comprises a nucleic acid construct comprising a gene encoding a Wnt activator or a Wnt modulator and a reporter gene regulated by a promoter modulated by the interaction between TCF/LEF and β-catenin, contacting the cell with a test compound, and identifying as a compound that modulates Wnt signaling a test compound that has the effect of modulating the signal from expression of the reporter gene in the cell contacted with the test compound with respect to the signal from expression of the reporter gene in a control cell not contacted with the test compound, in which the effect is not seen in cells in which the nucleic acid construct comprising the gene encoding the Wnt activator or Wnt modulator is not present.

In yet another aspect, a method is provided for identifying a compound that modulates Wnt signaling, comprising: providing a cell that comprises 1) a nucleic acid construct comprising a gene encoding a Wnt activator or a Wnt modulator under the control of an inducible promoter and 2) a reporter gene regulated by a promoter modulated by the interaction between TCF/LEF and β-catenin, inducing expression of the Wnt activator or Wnt modulator; contacting the cell with a test compound, and identifying a test compound that has the effect of modulating the signal from expression of the reporter gene in the cell contacted with the test compound with respect to the signal from expression of the reporter gene in a control cell not contacted with the test compound, in which the effect is not obtained in cells in which the gene encoding the Wnt activator or Wnt modulator is not induced.

In these aspects, the assay cells include a recombinant construct that includes a gene for a Wnt activator or a Wnt modulator. A Wnt activator is any protein that when expressed in the cell, modulates Wnt signaling. Nonlimiting examples of Wnt activators include β-catenin, APC, axin1, axin2, GSK3, Disheveled, LRP5, LRP6, Frizzled, or Wnt proteins. A Wnt modulator is any protein that when expressed in the cell, modulates Wnt signaling by regulating the expression of one or more Wnt activators or one or more Wnt modulators. Nonlimiting examples of Wnt modulators include β-catenin, TCF-1, TCF-2, TCF-3, TCF-4, as well as the transcriptional repressors that interact with TCF/LEF proteins or β-catenin, including: CtBP, Groucho, Pygo, p300, and PITX2. In some embodiments, a Wnt activator or modulator expressed in cells is a mutant form of the activator or modulator. In some embodiments, the Wnt activator is a mutant APC gene. In some embodiments, the Wnt activator is a mutant β-catenin gene.

A reporter gene used in the assays presented herein can be any reporter gene, such as, for example, an alkaline phosphatase, beta-galactosidase, beta-lactamase, a fluorescent protein, a luciferase, or CAT. In some preferred embodiments, the assay cells used in the methods include at least two reporter genes, at least one of which is a control reporter gene under the control of a promoter that is not regulated by the interaction of TCF/LEF and β-catenin, for example, a constitutive promoter. In these embodiments, the signal detected from expression of a reporter gene that is operably linked to a promoter modulated by the interaction between TCF/LEF and β-catenin is normalized to the signal detected from a second reporter gene whose expression is regulated by a constitutive promoter.

In a further aspect, the invention includes a method for identifying a compound that modulates Wnt signaling using negative selection to identify compounds that disrupt the activation of genes by the interaction of TCF/LEF and β-catenin. The method includes: providing a cell having a reporter gene regulated by a promoter modulated by the interaction between TCF/LEF and β-catenin, wherein the reporter gene is negatively selectable, contacting the cell with a test compound, contacting the cell with a prodrug that is converted to an active drug by the protein encoded by the reporter gene; and identifying a test compound that permits the growth of cells in the presence of the prodrug.

Also provided herein are compounds identified by any of one or more methods as described above.

### BRIEF DESCRIPTION OF THE DRAWINGS

FIG. 1 shows cancer-associated Wnt reporters.

FIG. 2 shows the human embryonic kidney cell line HEK293 is transiently transfected with: 1) a gene encoding β-catenin under the constitutive control of the cytomegalovirus (CMV) promoter and linked by an IRES to a gene encoding a red fluorescent protein, and 2) a reporter gene construct that includes the gene for green fluorescent protein (GFP) under the control of the axin2 promoter.

FIGS. 3A and 3B illustrate the promoter sequence for the naturally occurring Wnt responsive promoter SP₅ (from Naoko Fujimura et al. JBC 2007 which illustrates SP₅ promoter).

FIG. 4 illustrates the promoter sequence for the naturally occurring Wnt responsive promoter DKK4.

FIG. 5 shows the WinThunder which is a synthetic Wnt responsive DNA consisting of 12× or more WREs.

FIG. 6 shows how siRNA to β-catenin effectively downregulated β-catenin expression and abrogated WinBeam luciferase activity.

FIG. 7 shows how WinVerve luciferase activity is downregulated siRNA to β-catenin.

FIG. 8 shows how cWinThunder activity is downregulated by siRNA to β-catenin.

FIG. 9 shows SV40 promoter activity is not affected by β-cat siRNA.

FIG. 10 shows how WinBeam is constitutively active in SW480, HCT-116 and DLD1.

FIG. 11 shows how WinVerve is constitutively active in SW480 and HCT-116.

FIG. 12 shows WinThunder luciferase activity in SW480 and HCT-116 cells.

FIG. 13 shows how WinBeam is activated by β-catenin and a Wnt activator in 293 cells.

FIG. 14 shows how WinVerve is activated by a β-catenin and a Wnt activator in 293 cells.

FIG. 15 shows WinThunder activity in 293 Trex cells with inducible β-cat.

FIG. 16 shows compounds selected using WIN reporters.

FIG. 17 shows how non-specific and toxic compounds are excluded from analysis using WIN reporters.

FIG. 18 shows how toxic and non-specific compounds with no effect on the WIN reporters are excluded from analysis.

### DETAILED DESCRIPTION OF THE INVENTION

### Definitions

As used herein, a cancerous cell or cancer cell is a leukemia cell or a cell derived from a cancerous tumor. One test for whether a nonleukemia cell is cancerous is whether an inoculum of the cells in a nude mouse causes a tumor or tumors. As used herein, a “normal” cell is a noncancerous cell. A normal or noncancerous cell is not derived from a cancerous tumor or leukemia.

“Wnt signaling” or “Wnt pathway signaling” refers to a cell signaling pathway that results in the expression of genes regulated by the interaction of beta catenin with a TCF/LEF protein, such as TCF-1, TCF-3, TCF-4, or LEF-1.

A “protein that participates in Wnt signaling” or a “Wnt-related protein” can be a Wnt activator, a Wnt modulator, or a Wnt target gene. A “Wnt-related gene” is a gene that encodes a protein that participates in Wnt signaling. Proteins that participate in Wnt signaling include, without limitation, Wnt activators (proteins that promote or inhibit beta catenin-TCF/LEF interaction that leads to Wnt target gene expression), including Wnt, Frizzled, Disheveled, LRP5/LRP6 (BMC Genomics 7: 148 (2006)), axin-1 (BMC Genomics 7: 148 (2006)), beta-catenin (BMC Genomics 7: 148 (2006)), axin-2, adenomatous polyposis coli (APC), GSK3-beta (BMC Genomics 7: 148 (2006)); and Wnt modulators (proteins that modulate Wnt target gene expression), including TCF-1 (J. Biol. Chem. 267: 8530-8536 (1992); Mol. Cell. Biol. 16: 745-752 (1996), TCF-3, TCF-4 (J. Biol. Chem. 278: 16169-16175 (2003)); LEF-1 (Nucl. Acids Res. 28: 1994-2003 (2000); Devel. Dynamics 232: 969-978 (2005), CtBP1, Grouch, Pygo, PITX2, and others.

Wnt target genes include, without limitation LEF-1, c-myc, cyclin D1, cdx, MMP7, c-myb, c-kit, PPARsigma, axin2, Bcl-X, sp5, siamois, and others.

“TCF/LEF” refers to any one of TCF-1, TCF-3, TCF-4, or LEF-1, or any combination of two or more of TCF-1, TCF-3, TCF-4, or LEF-1.

As used herein a “Wnt-responsive promoter” is a promoter that is regulated by the interaction of a TCF/LEF protein and β-catenin. The promoter may be regulated by other factors in addition to a TCF/LEF protein and β-catenin. Examples of Wnt-responsive promoters include, but are not limited to, the promoters of the following genes: LEF-1, TCF1, c-myc, c-kit, MMP7, axin2, sp5, DKK4, cyclinD1, cdx, Bcl-X, and siamois.

As used herein, RNA “isoforms” or transcript isoforms or isoform transcripts are RNA molecules generated by alternative splicing of the same gene. The sequences of the transcript therefore differ. Protein isoforms are translated from RNA isoforms, and have different primary sequences.

“Nucleic acid molecule construct”, “Nucleic acid construct”, “gene construct”, “reporter gene construct”, “splicing construct”, “transcription construct”, “construct”, “recombinant DNA molecule” all refer to nucleic acid molecules that have been isolated and manipulated to excise, join, delete, mutate, expand, extend, replicate, or recombine certain nucleic acid sequences that may be isolated from organisms, replicated from nucleic acid templates isolated from organisms, synthesized, or derived from organisms and synthetic nucleic acid fragments. In the methods of the invention, cells that comprise, include, carry, or have nucleic acid molecules or nucleic acid constructs are cells that have been transformed, transfected, or infected (e.g., with a virus) such that they contain a previously isolated nucleic acid molecule or recombinant nucleic acid molecule or gene construct.

The methods provided herein are used to identify compounds that modulate Wnt signaling in cancer cells. The methods use cell-based assays in which the activity of reporter genes regulated by Wnt signaling-responsive promoters in response to test compounds are compared to the effects of test compounds on noncancerous cells, or to the effects of the test compounds on cells in which the Wnt signaling pathway is not activated by the introduction of induction of Wnt activators or Wnt modulators in the cells.

### Assay Formats

The cell-based assays provided herein can be performed in any feasible format, but are preferably high throughput assays for screening large numbers of compounds. Preferably, the assays are performed in multiwell dishes, such as, for example dishes with 96, 384, or more wells, where each well holds from about 5×10³ to 10⁵ cells, typically from about 10⁴ to 5×10⁴ cells. In preferred embodiments, the assays are performed using reporter genes, in which the signal from the reporter gene is detected by, for example, a luminometer or fluorometer that reads multiwell plates. Plate readers that include an automated dispensing device (for example, for adding reagent buffer for signal detection) are also preferred.

For assays in which cells are transiently transfected with reporter gene constructs or Wnt activator or modulator gene constructs, addition of test compound is typically added 24-48 hours after transfection. In assays in which expression of a gene is induced, for example, by addition of an inducer such as tetracycline or doxycycline, test compound can be added before, at the same time as, or after the inducer. For example, a test compound can be added from 0 to 30 minutes, from 30 minutes to one hour, from one to two hours, from two to three hours, from three to four hours, from four to six hours, from six to eight hours, from eight to ten hours, from 10 to 12 hours, from 12 to 16 hours, from 16 to 20 hours, from 20 to 24 hours, or from 24 to 48 hours after the addition of an inducer.

Reading of the reporter gene signal(s) can be at any time point after the addition of compound, for example, 30 minutes, between 30 minutes and one hour, between one and two hours, between two and three hours, between three and four hours, between four and six hours, between six and eight hours, between eight and ten hours, between 10 and 12 hours, between 12 and 16 hours, between 16 and 20 hours, between 20 and 24 hours, or between 24 and 48 hours after the addition of compound.

Test compounds may be used at a concentration of from about 10 picomolar to about 10 micromolar, for example, from about 1 nanomolar to about 1 micromolar. Initial screens may be performed at a concentration of, for example 100 nanomolar to 10 micromolar, and subsequent secondary screens can be performed at a higher or lower concentration, or at a range of concentrations.

Cellular assays can also be performed to determine the effect of test compounds on the metabolic state, proliferation, growth, or viability of the cells. One or more of a viability assay, cell division assay, cell cycle assay, migration assay, invasion assay, cell death assay, or apoptosis assay, can be performed on the cells in addition to the reporter gene readout assays described herein. For example, cell growth can be monitored using an MTT assay (e.g., the VYBRANT® MTT cell proliferation assay kit, Invitrogen Corp., Carlsbad, Calif.) or its derivative XTT assay (MD Biosciences, St. Paul, Minn.) or BrdU incorporation (the ABSOLUTE-S™ SBIP assay (Invitrogen Corp.). Cell viability (or cytotoxicity) can be assayed by measuring intracellular ATP levels (the ATPLITE™-M kit (Perkin Elmer) or the Cell Titer Glo (PromegaCorp., Madison, Wis.) or glucose-6-phosphate activity (the Vibrant cytotoxicity assay (Invitrogen Corp.) or by assays using a membrane permeable dye (DiOc 18). In some embodiments, cellular assays are performed in a separate secondary screen. In some embodiments, cellular assays are performed simultaneously with reporter gene assays. For example, assays for viability that use Alamar blue (Nasiry et al., Human Reprod 22: 1304-1309 (2007)) or assays for apoptosis that detect caspase activity (e.g., the APOALERT® caspase assay kits available from Clontech, Mountain View, Calif.), can be performed in the same wells in which reporter gene expression is assayed, provided that the cellular assay readout is distinguishable from the reporter gene expression readout.

### Cells

A cancerous cell used in the methods can be any cancerous cell, and can be, as nonlimiting examples, a colon cancer cell, a leukemia cell, a lymphoma cell, a melanoma cell, a breast cancer cell, a prostate cancer cell, a hepatocarcinoma cell, a lung cancer cell, an ovarian cancer cell, a uterine cancer cell, a cervical cancer cell, or a head-and-neck cancer cell. Nonlimiting examples of leukemia cells include Jurkat, HL60, and K562 cells. Nonlimiting examples of colon cancer cells include SW48, SW480, SW116, CaCo-2, DLD1, Colo320, Colo205, HT29, and HT116 cells.

A noncancerous cell used in the methods can be any cancerous cell, and can be, as nonlimiting examples, a HEK293 cell, a COS-7 cell, a CHO cell, a NIH/3T3 cell, or a noncancerous colon cell, noncancerous intestinal epithelial cell, epithelial cell, skin cell, B cell, pre-B cell, T cell, pre-T cell, breast cell, prostate cell, liver cell, lung cell, ovarian cell, or cervical cell. Noncancerous colon (intestinal epithelial) cells include, without limitation, NCM 356 cells and NCM 460 cells ((Stauffer et al., Amer. J. Surg. 169: 190-195 (1995); Battacharya et al., Amer. J. Gastr. Liv. Physiol. 293: G429-437 (2007); both available from Incell Corp.), and NCIEM cells (Baten et al., FASEB J. 6: 2726 (1992)). Noncancerous cells can be transformed with the T antigen of SV40 to improve their transfectability. Primary cells can be isolated and immortalized by stably transfecting the cells with the T antigen of SV40 or hTERT (WO 2003/010305).

### Reporter Genes

Reporter genes include any genes whose expression is detectable, for example, by detection of the protein itself (e.g., fluorescent proteins), affintiy-based detection of a domain of the protein (e.g., a peptide tag such as a flag tag or by expression of a peptide sequence that is a “self-labeling tag”, e.g., a FlASH or “lumio” tag that binds a fluorescent reagent) or by detecting the product of an enzymatic reaction catalyzed by the reporter protein

Fluorescent proteins include, without limitation, phycoerythrin, phycocyanin, allophycocyanin, a green fluorescent protein, a yellow fluorescent protein, a red fluorescent protein, an orange fluorescent protein, a cyan fluorescent protein, or a blue fluorescent protein. The variety of fluorescent proteins with different excitation and emissions spectra make them particularly useful where two or more reporter genes are desirable. Lentiviral vectors designed to investigate the expression of several genes in parallel in a single cell have been used to introduce three differently detectable fluorescent proteins in separate viral constructs into the same cell (Weber et al. Mol Ther. 16: 698-706 (2008)). Fluorescent protein detection is non-invasive, and may be done repeatedly on a same sample over time. Fluorescent protein genes used in the methods of the invention can be mutant forms of fluorescent protein genes. For example, the fluorescent protein genes can be mutants that are humanized or have enhanced fluorescence with respect to wild type proteins, or can be mutants with a higher turnover such that reporter gene measurements more accurately reflect a dynamic process such as changes in splicing or gene expression patterns in response to a modulating compound.

Enzymes that convert substrates to detectable products include alkaline phosphatase, beta galactosidase, beta lactamase, and luciferases. For example, substrates of alkaline phosphatase, beta galactosidase, beta lactamase can be conjugates that produce fluorescent compounds when cleaved. In some embodiments, secreted forms of these enzymes may be used.

Luciferases that can be used in the methods of the invention include, without limitation, beetle luciferases (including click beetle and firefly luciferases), Renilla luciferase, and Gaussia luciferase (Verhaegeb et al. Anal. Chem. 74: 4378-4385 (2002); Tannous et al. Mol. Ther. 11: 435-443 (2005)). Luciferase assays are quantitative and exhibit very low background. With the exception of the secreted Gaussia luciferase, luciferase assays generally require lysis of the assay cell. In some embodiments, however, a membrane-permeable luciferase reagent may be used, obviating cell lysis. Luciferases having different emissions optima can be used in two-reporter gene assays. For example, firefly luciferase and Renilla luciferase have distinguishable signals, and assay buffers are available that allow the signal from the two luciferases to be read in tandem (Promega Corp., Madison, Wis.). Click beetle red and green luciferase mutants have also been designed to have distinct emission spectra, so that two click beetle luciferase reporter genes can be used in the same assay using the same substrate buffer (Promega Corp., Madison, Wis.).

### Wnt Modulators and Activators

In some embodiments, noncancerous or cancerous cells used in the methods of the invention also include a recombinant construct that includes a gene for a Wnt activator or a Wnt modulator. A Wnt activator is any protein that when expressed in the cell, modulates Wnt signaling. Nonlimiting examples of Wnt activators include β-catenin, APC, axin1, axin2, GSK3, Disheveled, LRP5, LRP6, Frizzled, or Wnt proteins. A Wnt modulator is any protein that when expressed in the cell, modulates Wnt signaling by regulating the expression of one or more Wnt activators or one or more Wnt modulators. Nonlimiting examples of Wnt modulators include β-catenin, TCF-1, TCF-2, TCF-3, TCF-4, as well as the transcriptional repressors that interact with TCF/LEF proteins or β-catenin, including: CtBP, Groucho, Pygo, p300, and PIX2. In some embodiments, a Wnt activator or modulator expressed in cells is a mutant form of the activator or modulator. In some embodiments, the Wnt activator is a mutant APC gene. In some embodiments, the Wnt activator is a mutant β-catenin gene.

### Gene Transfer and Vectors

The recombinant reporter gene constructs or constructs for expression of Wnt modulators or activators that are used in the assay methods can be transiently transfected into cells, or can be integrated into the host cell. For transient transfection or selection of stable integrants, recombinant reporter gene constructs are introduced into cells as plasmids. Nucleic acid constructs can be transfected into cells using any methods for introducing DNA into cells, including, for example, electroporation, DNA biolistics, lipid-mediated transfection, compacted DNA-mediated transfection, liposomes, dextran, immunoliposomes, lipofectin, cationic agent-mediated transfection, cationic facial amphiphiles (CFAs) (Nature Biotechnology 1996 14; 556), multivalent cations such as spermine, cationic lipids or polylysine, 1,2,-bis(oleoyloxy)-3-(trimethylammonio)propane (DOTAP)-cholesterol complexes (Woff and Trubetskoy 1998 Nature Biotechnology 16: 421) and combinations thereof. Selection of stable integrants is typically by selection on media containing an antibiotic for which the plasmid that includes the reporter gene construct has a resistance gene.

In some preferred embodiments of the invention, the reporter gene constructs or Wnt activator or Wnt modulator constructs are introduced into the cell using viral vectors delivery systems. For example, the nucleic acid constructs can be introduced into cells using adenoviral vectors, adeno-associated viral (AAV) vectors, herpes viral vectors, or retroviral vectors (including lentiviral vectors). Viral vectors delivery system provide the advantages of random and stable integration, the ability to transducer cells that may be otherwise recalcitrant to gene delivery methods, and single site integration of recombinant genes, providing a more reliable and consistent assay system. Inducible viral expression vectors include, for example, those disclosed in U.S. Pat. No. 6,953,575.

Retroviruses that can be used to reporter gene constructs and Wnt activator or modulator genes into cells include, without limitation: murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV) and lentiviruses, which have the ability to infect both dividing and non-dividing cells.

Examples of primate lentiviruses include the human immunodeficiency virus (HIV), and simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV).

More than one retrovirus (or lentivirus) can be used to infect the same cell, providing the possibility of using retroviral vectors for introducing more than one reporter gene construct, Wnt modulator gene, Wnt activator gene, and combinations thereof. Infection of cells with three retroviruses can be done simultaneously, by infecting the cells with a mixture of the different engineered viruses, and selecting for cells carrying each of them (Weber et al. Mol Ther. 16: 698-706 (2008)).

### Test Compounds

Test compounds can be small molecules, peptides, polypeptides, carbohydrates, lipids, or nucleic acid molecules. A test compound can be a member of a library of natural or synthetic compounds. For example, test compounds can be from a combinatorial library, i.e., a collection of diverse chemical compounds generated by either chemical synthesis or biological synthesis by combining a number of chemical building blocks.

Test compounds can also include polypeptides and peptides, including peptide mimetics based on polypeptides. Test compounds can also be nucleic acid aptmers, nucleic acid molecule “decoys” of transcriptional promoter or enhancer sequences or splicing junctions or enhancers. In some embodiments, test compounds can be in the form of nucleic acid constructs that induce triple helical structures to inhibit transcription of a gene (Helene (1991) Anticancer Drug Des. 6:569-584).

In some embodiments, test compounds can include RNAi constructs or antisense oligonucleotides directed against one or more isoforms of a Wnt activator or modulator or components of the Wnt signaling pathway. In some embodiments, a test compound is a nucleic acid molecule that comprises one or more ribozymes directed against one or more isoforms of genes that participate in Wnt signaling. The design, synthesis, and use of RNAi constructs, antisense oligonucleotides, and ribozymes are found, for example, in Dykxhoorn et al. (2003) Nat. Rev. Mol. Cell. Biol. 4: 457-467; Hannon et al. (2004) Nature 431: 371-378; Sarver et al. (1990) Science 247:1222-1225; Been et al. (1986) Cell 47:207-216).

For example, a test compound in some embodiments is an siRNA (“short interfering RNA”) molecule or a nucleic acid construct that produces an siRNA molecule. In some embodiments, test compounds are introduced into the cells as one or more short hairpin RNAs (“shRNAs”) or as one or more DNA constructs that are transcribed to produce one or more shRNAs, in which the shRNAs are processed within the cell to produce one or more siRNA molecules.

Nucleic acid constructs for the expression of siRNA, shRNA, antisense RNA, ribozymes, or nucleic acids for generating triple helical structures are optionally introduced as RNA molecules or as recombinant DNA constructs. DNA constructs for reducing gene expression or splicing of particular isoforms are optionally designed so that the desired RNA molecules are expressed in the cell from a promoter that is transcriptionally active in mammalian cells. For some purposes, it is desirable to use viral or plasmid-based nucleic acid constructs to introduce the test compounds.

### Pharmaceutical Compositions and Methods of Administration

Pharmaceutical compositions are formulated using one or more physiologically acceptable carriers including excipients and auxiliaries which facilitate processing of the active compounds into preparations which are used pharmaceutically. Proper formulation is dependent upon the route of administration chosen. A summary of pharmaceutical compositions is found, for example, in Remington: The Science and Practice of Pharmacy, Nineteenth Ed (Ea hston, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

Provided herein are pharmaceutical compositions that include one or more compounds that modulates Wnt signaling (a “Wnt signaling modulator) and a pharmaceutically acceptable diluent(s), excipient(s), or carrier(s). In addition, the Wnt signaling modulator is optionally administered as pharmaceutical compositions in which it is mixed with other active ingredients, as in combination therapy. In some embodiments, the pharmaceutical compositions includes other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions also contain other therapeutically valuable substances.

A pharmaceutical composition, as used herein, refers to a mixture of a Wnt signaling modulator with other chemical components, such as carriers, stabilizers, diluents, dispersing agents, suspending agents, thickening agents, and/or excipients. The pharmaceutical composition facilitates administration of the Wnt isoform expression modulator to an organism. In practicing the methods of treatment or use provided herein, therapeutically effective amounts of a Wnt signaling modulator are administered in a pharmaceutical composition to a mammal having a condition, disease, or disorder to be treated. In some embodiments, the disease is cancer. Preferably, the mammal is a human. A therapeutically effective amount varies depending on the severity and stage of the condition, the age and relative health of the subject, the potency of the Wnt signaling modulator used and other factors. The Wnt signaling modulator is optionally used singly or in combination with one or more therapeutic agents as components of mixtures.

The pharmaceutical formulations described herein are optionally administered to a subject by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

The pharmaceutical compositions in some embodiments will include at least one Wnt isoform expression modulator, as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these Wnt isoform expression modulator having the same type of activity. In some situations, Wnt isoform expression modulators exist as tautomers.

“Carrier materials” include any commonly used excipients in pharmaceutics and should be selected on the basis of compatibility with compounds disclosed herein, such as, a Wnt isoform expression modulator, and the release profile properties of the desired dosage form. Exemplary carrier materials include, e.g., binders, suspending agents, disintegration agents, filling agents, surfactants, solubilizers, stabilizers, lubricants, wetting agents, diluents, and the like.

The pharmaceutical compositions described herein, which include a Wnt signaling modulator, are formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

For administration by inhalation, the Wnt signaling modulator is optionally in a form as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit is determined by providing a valve to deliver a metered amount. Capsules and cartridges of, such as, by way of example only, gelatin for use in an inhaler or insufflator are formulated containing a powder mix of the Wnt isoform expression modulator and a suitable powder base such as lactose or starch.

Transdermal formulations of a Wnt signaling modulator are administered for example by those described in U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144.

Formulations that include a Wnt signaling modulator suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

For intravenous injections, a Wnt signaling modulator is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, the pharmaceutical composition described herein are in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of the Wnt signaling modulator in water soluble form. Additionally, suspensions of the Wnt signaling modulator are optionally prepared as appropriate oily injection suspensions.

In some embodiments, the Wnt signaling modulator is administered topically and formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

The Wnt signaling modulator is also optionally formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like.

Assays for Identifying Compounds that Modulate Wnt Signaling in a Cancerous Cell

Methods are provided for identifying a compound that modulates Wnt signaling in a cancerous cell, in which the method includes: providing a cancerous cell that comprises a reporter gene regulated by a promoter modulated by the interaction between TCF/LEF and β-catenin, providing a noncancerous cell that comprises the reporter gene regulated by a promoter modulated by the interaction between TCF/LEF and β-catenin, contacting the cancerous cell and the noncancerous cell with a test compound, and identifying a test compound that modulates the signal from expression of the reporter gene in the cancerous cell, but does not modulate the signal from expression of the reporter gene in the non-cancerous cell. Modulation of the signal from the reporter gene in a cell is determined by comparing a signal detected for the cell when the cell is contacted with the test compound to a signal detected when the cell is not contacted with the test compound.

A cancerous cell used in the methods can be any cancerous cell, and can be, as nonlimiting examples, a colon cancer cell, a leukemia cell, a lymphoma cell, a melanoma cell, a breast cancer cell, a prostate cancer cell, a hepatocarcinoma cell, a lung cancer cell, an ovarian cancer cell, a cervical cancer cell, or a head-and-neck cancer cell. In some embodiments, the cancerous cell is a leukemia cell, for example, a Jurkat cell, an HL60 cell, or a K562 cell. In some embodiments, the cancerous cell is a colon cancer cell, for example, an SW116 cell, an SW48 cell, an SW480 cell, a Colo 320 cell, a Colo 205 cell, an HT-29 cell, an HT-116 cell, or a CaCO2 cell.

A noncancerous cell used in the methods can be any cancerous cell, and can be, as nonlimiting examples, a HEK293 cell, a COS-7 cell, a CHO cell, a NIH/3T3 cell, or a noncancerous colon cell, epithelial cell, skin cell, B cell, T cell, breast cell, prostate cell, liver cell, lung cell, ovarian cell, uterine cell, or cervical cell. Noncancerous colon cells include, without limitation, NCM 356 cells and NCM 460 cells (both available from Incell Corp.), and NCIEM cells (Baten et al., FASEB J. 6: 2726 (1992))

In some embodiments, the methods are performed using a cancerous colon cell and a noncancerous colon cell.

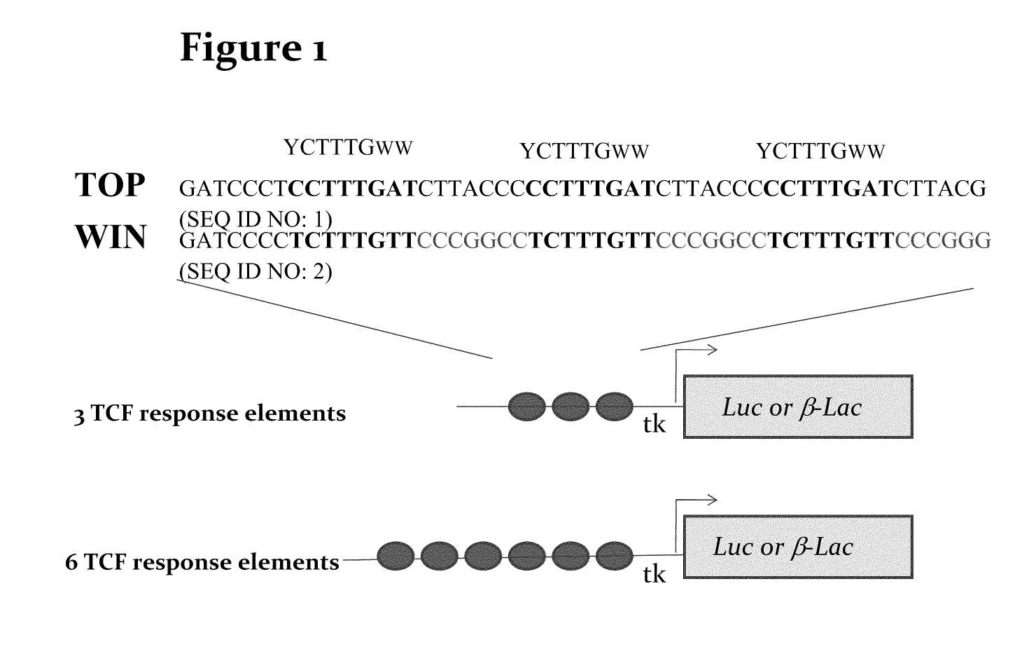

Promoters modulated by the interaction between TCF/LEF and β-catenin are any promoters that are upregulated, downregulated, repressed, or activated by a TCF/LEF protein bound to or acted on by β-catenin, including synthetic promoters, naturally-occurring promoters, portions of naturally-occurring promoters, chimeric promoters, etc. Also included are WIN promoters. A WIN promoter is a synthetic promoter encompassing one or more Wnt response elements (WREs) that are found in promoters of genes activated by the Wnt/b-catenin pathway or genes that are over-expressed in cancerous cells where the Wnt/b-catenin pathway is constitutively activated. Promoters useful in the expression constructs provided herein include WIN promoters, natural promoters of genes activated by the Wnt/b-catenin pathway encompassing at least two Wnt-response elements; a combination of natural promoters of genes with at least 2 or more Wnt-response elements combined with synthetic sequences flanking the Wnt-response elements; and natural promoters with two or more Wnt-response elements, combined with additional binding sites for other transcriptional activators. A synthetic promoter useful in the methods is the WIN promoter that includes the Wnt responsive elements also present in the “TOPFlash” promoter, but which includes GC-rich regions interspersed between the WREs (FIG. 1). FIG. 1 shows cancer-associated Wnt reporters. Synthetic promoters can include one or more GC-rich regions in addition to one or more Wnt-response elements found in promoters of genes that are activated by the Wnt/b-catenin pathway or genes that are over-expressed in cancerous cells where the Wnt/b-catenin pathway is constitutively activated. Also useful in the constructs are natural promoters of genes activated by the Wnt/b-catenin pathway encompassing at least two (or more) Wnt-response elements; a combination of natural promoters of genes with at least 2 or more Wnt-response elements combined with synthetic sequences flanking the Wnt-response elements; and natural promoters with two or more Wnt-response elements, combined with additional binding sites for other transcriptional activators.

Nonlimiting examples of naturally occurring promoters regulated by interaction between TCF/LEF and β-catenin include but are not limited to, the promoter of LEF-1, c-myc, c-myb, c-kit, CyclinD1, cdx, MMP7, survivin, Siamois, axin2, DKK4 and sp5.

Without limiting the methods of the invention or Wnt modulating compounds identified using the methods of the invention to any particular mechanism, it is understood that some promoters activated by binding of a TCF/LEF protein complexed with beta catenin may be activated by the interaction of beta catenin with a particular member of the TCF/LEF family and not another. A TCF/LEF-beta catenin regulated promoters can be regulated by additional proteins that may or may not interact with TCF/LEF or beta catenin. For example, LEF-1 is regulated by TCF-4 and not by LEF-1 itself, unless PITX2 is present, in which case LEF-1 interacts with PITX2 to induce its own transcription (Amen et al. Mol Cell. Biol. 27: 7560-7573). TCF-4E regulates LEF-1 and also cdx, which is not regulated by LEF-1. LEF-1 activates Siamois, whereas TCF-4E does not (Hecht et al. J. Biol. Chem. 278: 3776-3785 (2003)). The methods include embodiments in which the assays for identifying compounds that modulate Wnt signaling assay expression of two or more TCF/LEF-beta catenin responsive promoters, to ensure the effect of a compound is Wnt-pathway specific. For example, the cell to be assayed can have a first reporter gene operably linked to the WIN promoter, and a second reporter gene operably linked to the axin2 promoter. A third reporter gene can be operably linked to a constitutive promoter for normalizing reporter gene expression levels. In these embodiments, test compounds identified as Wnt pathway modulators are compounds that result in a difference in the expression of one or both genes in cancerous cells, but not in normal cells.

Also provided is a method of in which the method includes: providing a cell that comprises a nucleic acid construct comprising a gene encoding a Wnt activator or a Wnt modulator and a reporter gene regulated by a promoter modulated by the interaction between TCF/LEF and β-catenin, contacting the cell with a test compound, and identifying as a compound that modulates Wnt signaling a test compound that has the effect of modulating the signal from expression of the reporter gene in the cell contacted with the test compound with respect to the signal from expression of the reporter gene in a control cell not contacted with the test compound, in which the effect is not seen in cells in which the nucleic acid construct comprising the gene encoding the Wnt activator or Wnt modulator is not present.

In these aspects, the assay cells include a recombinant construct that includes a gene for a Wnt activator or a Wnt modulator. A Wnt activator is any protein that when expressed in the cell, stimulates or inhibits Wnt signaling. Nonlimiting examples of Wnt activators include β-catenin, APC, axin1, axin2, GSKβ3, Disheveled, LRP5, LRP6, Frizzled, or Wnt proteins. A Wnt modulator is any protein that when expressed in the cell, modulates Wnt signaling by regulating the expression of one or more Wnt activators or one or more Wnt modulators. Nonlimiting examples of Wnt modulators include β-catenin, TCF-1, TCF-2, TCF-3, TCF-4, as well as the transcriptional repressors and enhancers that interact with TCF/LEF proteins or β-catenin, including: CtBP, Groucho, Pygo, p300, and PIX2. In some embodiments, a Wnt activator or modulator expressed in cells is a mutant form of the activator or modulator. In some embodiments, the Wnt activator is a mutant APC gene. In some embodiments, the Wnt activator is a mutant β-catenin gene.

In yet another aspect, a method is provided for identifying a compound that modulates Wnt signaling, comprising: providing a cell that comprises 1) a nucleic acid construct comprising a gene encoding a Wnt activator or a Wnt modulator under the control of an inducible promoter and 2) a reporter gene regulated by a promoter modulated by the interaction between TCF/LEF and β-catenin, inducing expression of the Wnt activator or Wnt modulator; contacting the cell with a test compound, and identifying a test compound that has the effect of modulating the signal from expression of the reporter gene in the cell contacted with the test compound with respect to the signal from expression of the reporter gene in a control cell not contacted with the test compound, in which the effect is not obtained in cells in which the gene encoding the Wnt activator or Wnt modulator is not induced.

A reporter gene used in the assays presented herein can be any reporter gene, such as, for example, an alkaline phosphatase, beta-galactosidase, beta-lactamase, a fluorescent protein, a luciferase, or CAT. In some preferred embodiments, the assay cells used in the methods include at least two reporter genes, at least one of which is a control reporter gene under the control of a promoter that is not regulated by the interaction of TCF/LEF and β-catenin, for example, a constitutive promoter, In these embodiments, the signal detected from expression of a reporter gene that is operably linked to a promoter modulated by the interaction between TCF/LEF and β-catenin is normalized to the signal detected from a second reporter gene whose expression is regulated by a constitutive promoter.

The cells having a recombinant Wnt activator or modulator can be cancerous or noncancerous cells. In preferred embodiments of the methods in which a gene encoding a Wnt activator or modulator is provided in the cells to be assayed, the cells to be assayed are noncancerous cells in which the Wnt signaling pathway is activated by expression of the Wnt modulator or activator. Activation of the Wnt signaling pathway by the presence of or induction of expression of a Wnt activator or modulator can be assessed by the expression of the reporter gene having the Wnt-responsive promoter. Reporter gene expression that is at least two fold that of cells of the same type that lack the Wnt activator or modulator, or that are not induced to express the Wnt activator or modulator, indicates that the cells have an activated Wnt signaling pathway.

The noncancerous cells can be any cells, for example, HEK cells, COS-7 cells, NI|H/3T3 cells, CHO cells, etc., but are preferable noncancerous T cells, pre-T cells, breast cells, prostate cells, epithelial cells, colon cells, intestinal epithelial cells, skin cells, hepatocytes, lung cells, ovarian cells, uterine cells, or cervical cells, in which expression of a Wnt modulator or activator in these cells activates the Wnt signaling pathway. For example, noncancerous colon or intestinal epithelial cells such as but not limited to NCM 356 cells, NCM 460 cells, and NCIEM cells can be used in the methods presented herein.

A Wnt activator or Wnt modulator expressed in the assay cells can by any Wnt activator or modulator that activates the Wnt pathway. In some embodiments, the Wnt activator or modulator is a Wnt modulator, such as, for example, modulator comprises LEF1, TCF1, TCF3, TCF4, CtBP, Pygo, Groucho, CtBP, p300, or a truncated or mutant form thereof. In some embodiments the Wnt modulator expressed in the assay cells is LEF1, TCF1, TCF3, or TCF4, or a truncated or mutant form thereof. In some embodiments a recombinant Wnt modulator expressed in the assay cell is TCF-4E or LEF-1. In some embodiments, the Wnt activator or modulator is beta catenin, or a mutant or truncated form of a Wnt activator, such as, for example, a mutant form of β-catenin, APC, axin1, axin2, GSK3β, Disheveled, LRP5, LRP6, Frizzled, or Wnt, where the mutant form activates the Wnt pathway. In certain embodiments, the Wnt activator or modulator is Wnt, such as Wnt 1, Wnt 3, or Wnt 3a. In certain embodiments, the Wnt activator or modulator is Frizzled (Fz), such as Fz1, Fz3, Fz5, or Fz7.

In certain embodiments, the Wnt activator or modulator expressed in the assay cells in a mutant form of beta catenin that activates Wnt signaling, such as a mutant of beta catenin that lacks exon 3 phosphorylation sites, or a mutant form of APC that activates Wnt signaling, such as a truncated APC that lacks a functional beta catenin binding domain.

In some embodiments of the invention, the assay cells include nucleic acid constructs encoding two or more Wnt activators or modulators. The assay cells can express any combination of recombinant Wnt modulators and activators. In some embodiments of the invention, a Wnt modulator and a Wnt activator is introduced into the assay cells. For example, the assay cells can include recombinant constructs encoding beta catenin and LEF-1, or TCF-4 and a truncated APC gene, or LEF-1 and TCF-4. In preferred embodiments of the invention, the two or more Wnt modulators or activators introduced into the assay cells activate the Wnt pathway.

In a further aspect, the invention includes a method for identifying a compound that modulates Wnt signaling using negative selection to identify compounds that disrupt the activation of genes by the interaction of TCF/LEF and β-catenin. The method includes: providing a cell having a reporter gene regulated by a promoter modulated by the interaction between TCF/LEF and β-catenin, wherein the reporter gene is negatively selectable, contacting the cell with a test compound, contacting the cell with a prodrug that is converted to an active drug by the protein encoded by the reporter gene; and identifying a test compound that permits the growth of cells in the presence of the prodrug.

In these embodiments, cells that survive the negative selection are cells in which the Wnt pathway has been disrupted.

Reporter genes that can be used for negative selection include, without limitation, thymidine kinase, which converts acyclovir and gangcyclovir to toxic compounds, beta lactamase, which converts cephalosporin conjugates of drugs (such as for example, C-Dox [cepaholosporin doxorubicin and CCM [7-(4-carboxybutanamido)-cephalosporin mustard]) to their toxic form, cytosine deaminase for converting 5-fluorocytosine into 5-fluorouracil, alkaline phsophatase, which can convert phosphate containing prodrugs to their toxic form, arylsulfatase for converting sulfate containing prodrugs into free drugs, beta galactosidase and neuraminidase for converting glycosylated prodrugs into free drugs, and peptidases for converting peptide-containing prodrugs into free drugs.

### Example 1

### Cell-Based Assay for Modulation of Wnt Signaling Pathway in Cancer Cells

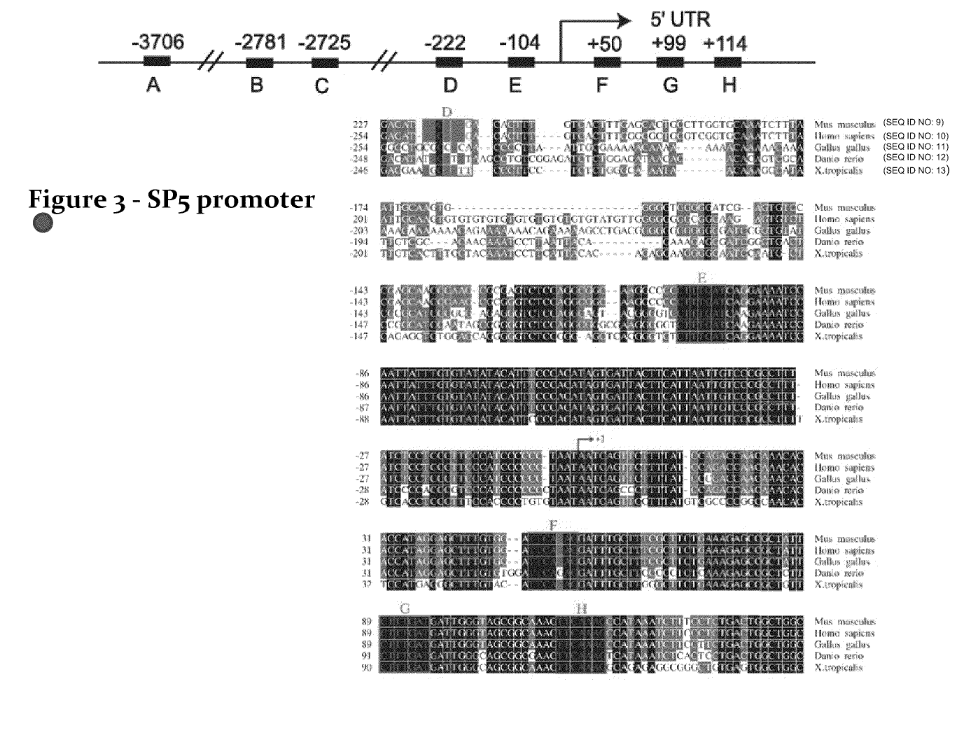

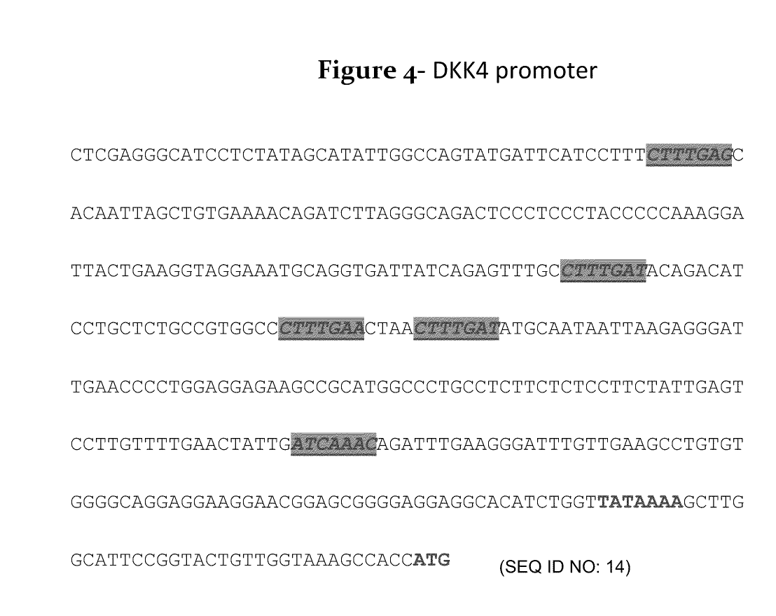

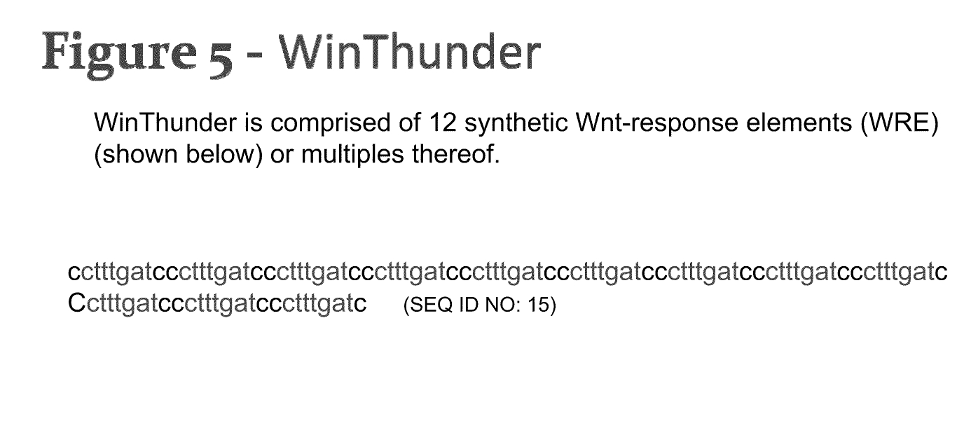

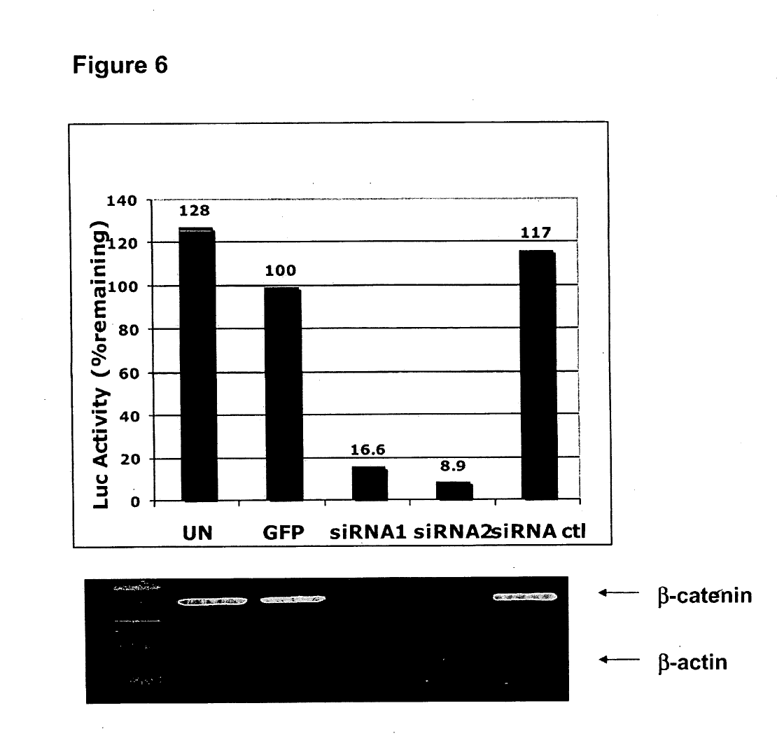

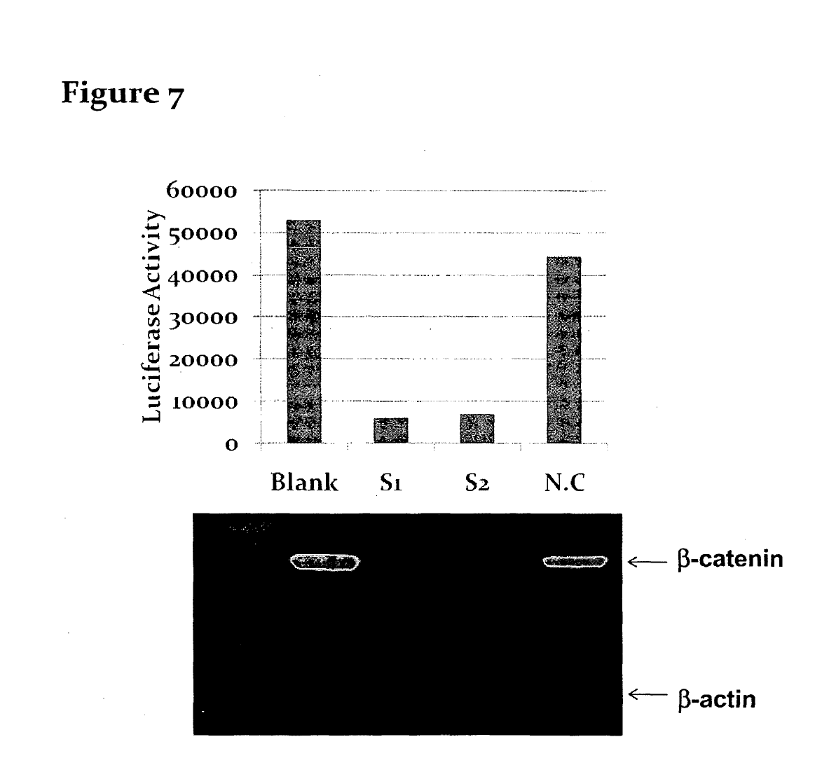

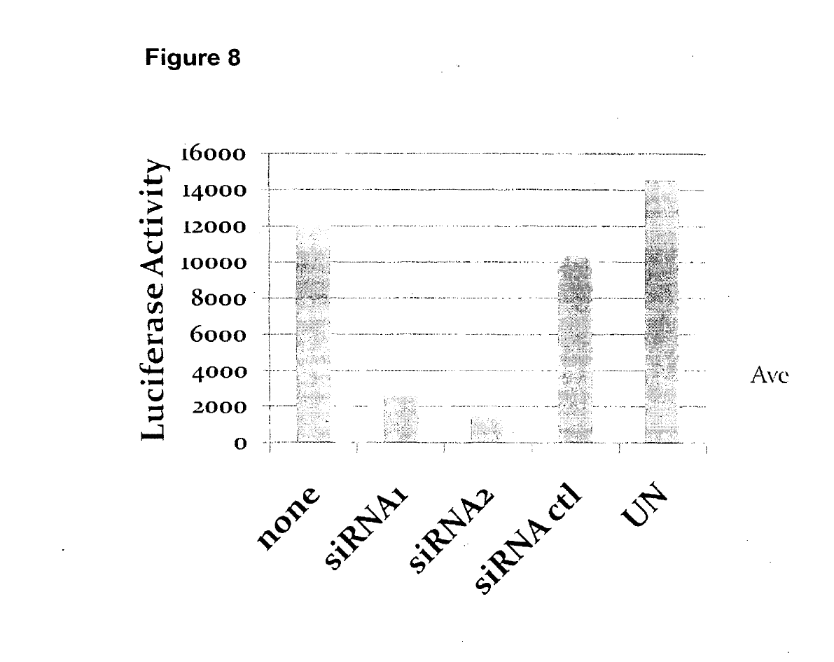

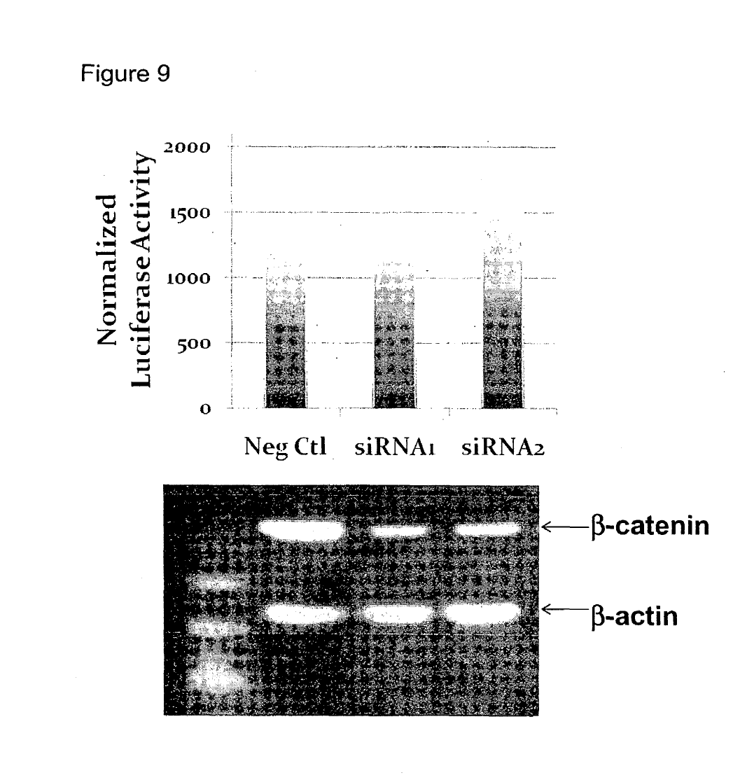

SW480 is a colon cancer cell line having a mutated APC gene that generates a truncated APC protein, leading to de-regulated accumulation of β-catenin. A reporter cell line, cWinBeam-SW480, is generated by stably transforming a cell line with a lentiviral vector containing the firefly luciferase gene under the control of a WIN promoter, WinBeam, which is based on the promoter sequence for the naturally occurring Wnt responsive promoter SP5 (FIG. 3 from Naoko Fujimura et al. JBC 2007 illustrates SP₅ promoter). Additionally, 2 other reporter cell lines, cWinVerve-SW480 and cWinThunder-SW480, were generated by stable transduction of SW480 with the firefly luciferase gene under the control of the WIN reporters, WinVerve, which is based on the promoter sequence for the naturally occurring Wnt responsive promoter DKK4 (FIG. 4), and WinThunder which is a synthetic Wnt responsive DNA consisting of 12× or more WREs (FIG. 5). In the cell lines generated, the WIN promoter luciferase activities were abrogated when siRNA to β-catenin, but not siRNA negative control, were introduced into the cells via lipid transfection (FIGS. 6-8). FIG. 6 shows how siRNA to β-catenin effectively downregulated β-catenin expression and abrogated WinBeam luciferase activity. FIG. 7 shows how WinVerve luciferase activity is downregulated siRNA to β-catenin. FIG. 8 shows how cWinThunder activity is downregulated by siRNA to β-catenin. Using the same cell line, a control reporter is generated by stable transduction of the firefly luciferase gene under the control of a constitutive promoter, SV40, which is not affected by siRNA to β-catenin. (FIG. 9). FIG. 9 shows SV40 promoter activity is not affected by β-cat siRNA.

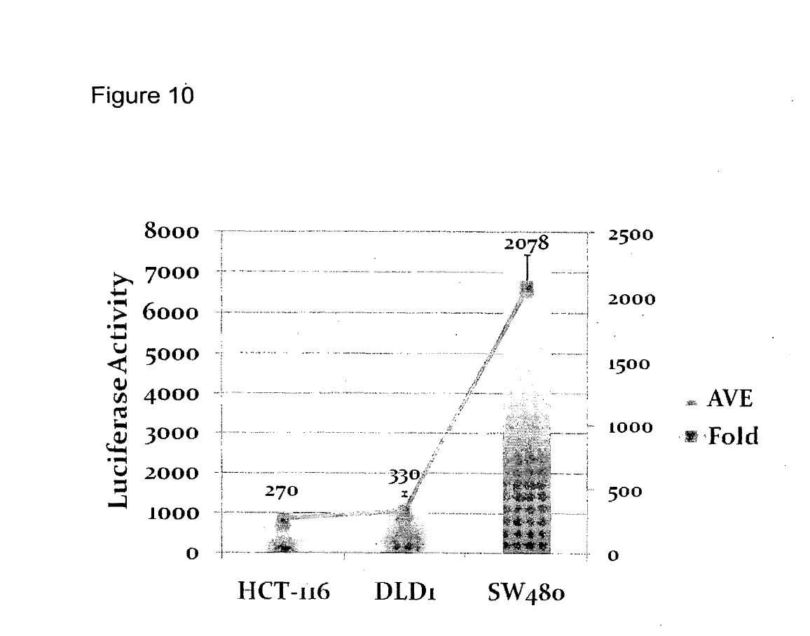

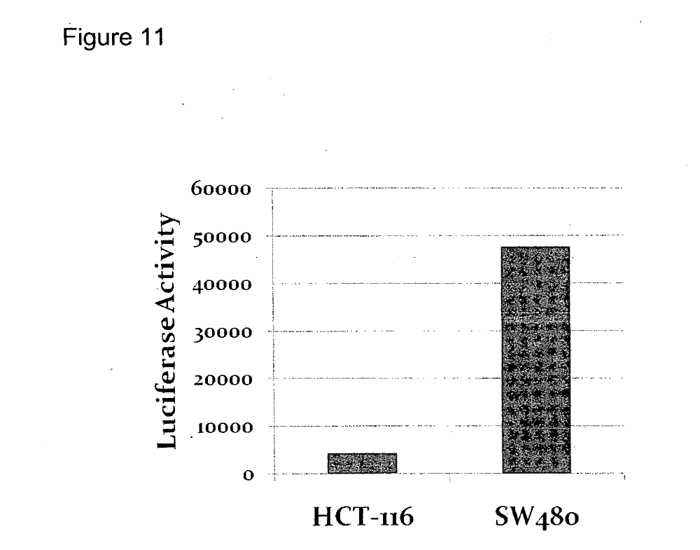

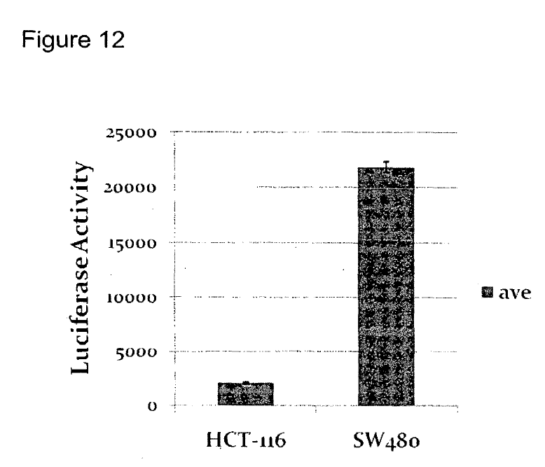

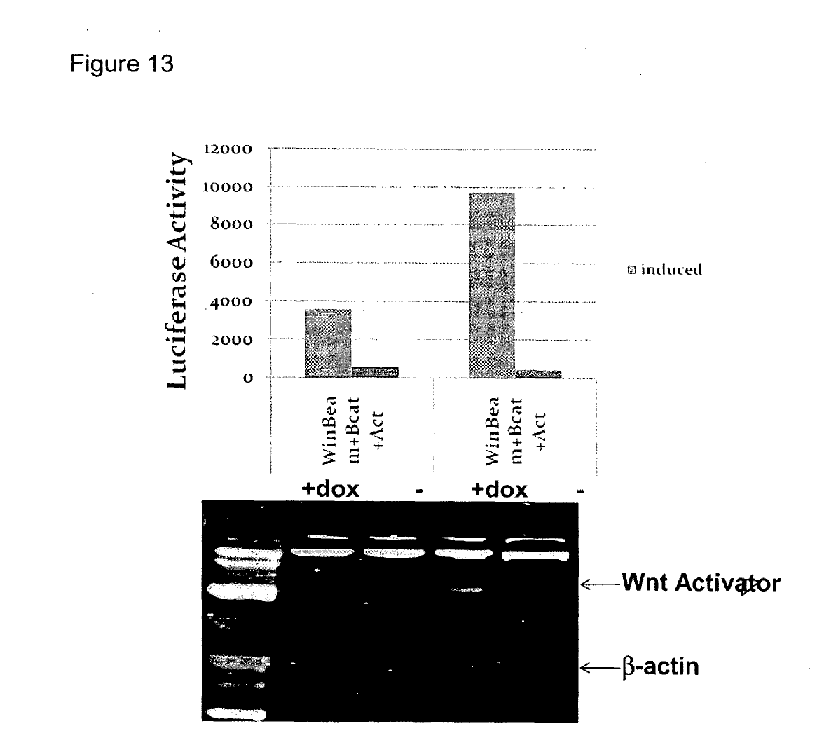

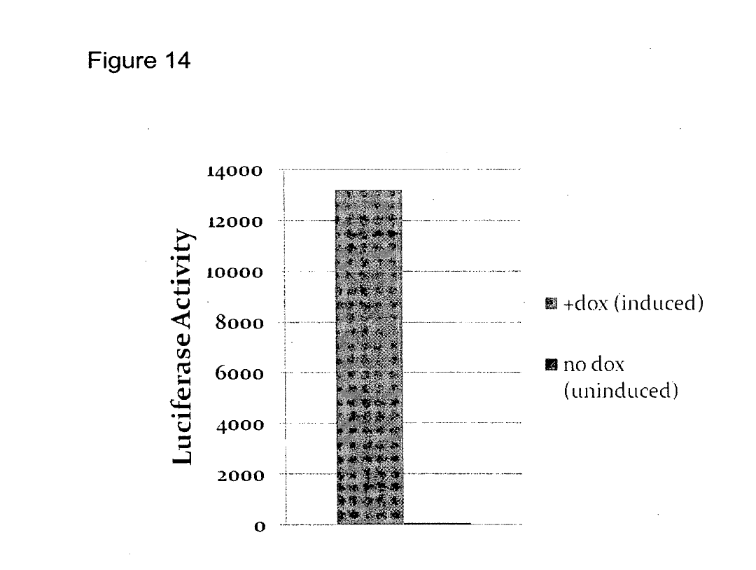

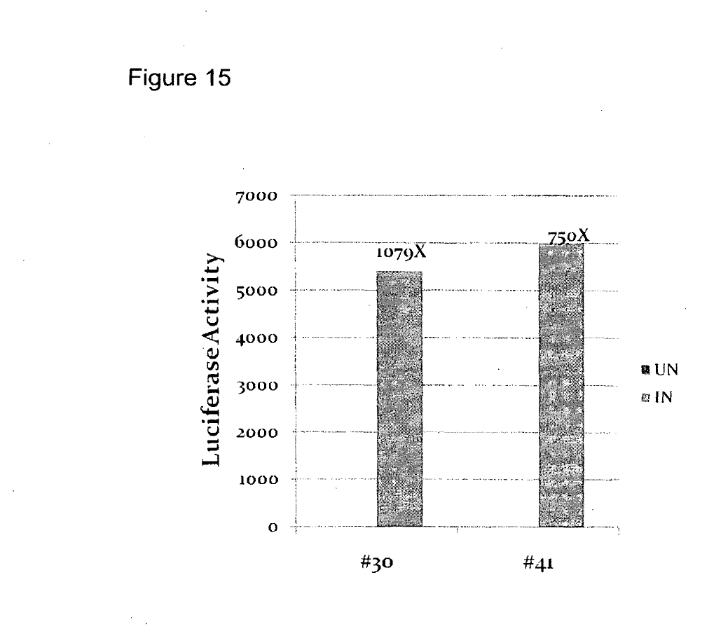

In some embodiments, other colon cancer cells with different mutations, like mutations in β-catenin, HCT-116, were also used to generate stable reporter cell lines with the WIN promoters: cWinBeam-HCT-116, cWinVerve-HCT-116 and cWinThunder-HCT-116 and control reporter cells with SV40 (FIGS. 10-12). FIG. 10 shows how WinBeam is constitutively active in SW480, HCT-116 and DLD1. FIG. 11 shows how WinVerve is constitutively active in SW480 and HCT-116. FIG. 12 shows WinThunder luciferase activity in SW480 and HCT-116 cells. In other embodiments normal cells such as the human embryonic kidney cell line HEK293 were also used to generate stable reporter cell lines with the WIN promoters: cWinBeam-293, cWinVerve-293 and cWinThunder-293 and control reporter cells with SV40 (FIGS. 13-15). FIG. 13 shows how WinBeam is activated by β-catenin and a Wnt activator in 293 cells. FIG. 14 shows how WinVerve is activated by a β-catenin and a Wnt activator in 293 cells. FIG. 15 shows WinThunder activity in 293 Trex cells with inducible β-cat.

In the case of normal cells, reporter activity is induced by either the addition of exogenous Wnt activators or stable expression of a gain-of-function “E3” mutant of β-catenin linked to the inducible “Tet-On” promoter. This mutant β-catenin lacks the exon 3 domain that includes phosphorylation sites S33, S37, T41, and S45, and therefore is not targeted for destruction by the β-catenin destruction complex, leading to constitutive Wnt signaling in the cell. The “Tet-On” promoter permits titratable induction by the addition of tetracycline or the tetracycline derivative doxycycline to cultures stable expression of a active b-catenin under the control of a antibiotic (FIGS. 13-15).

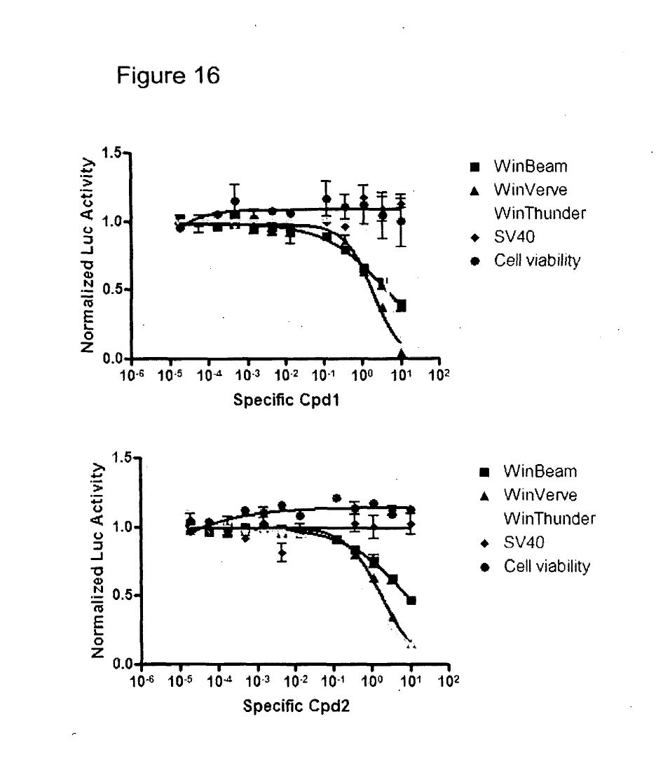

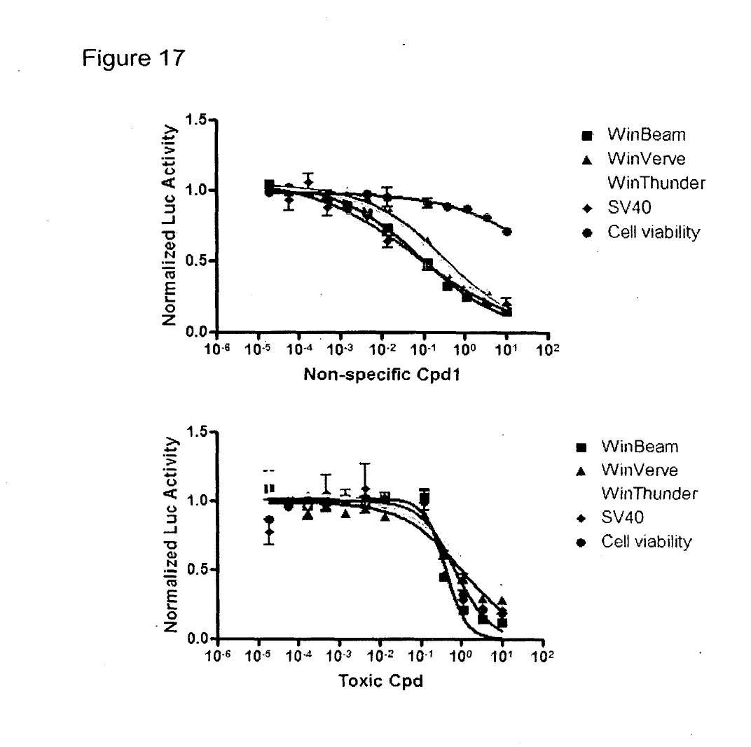

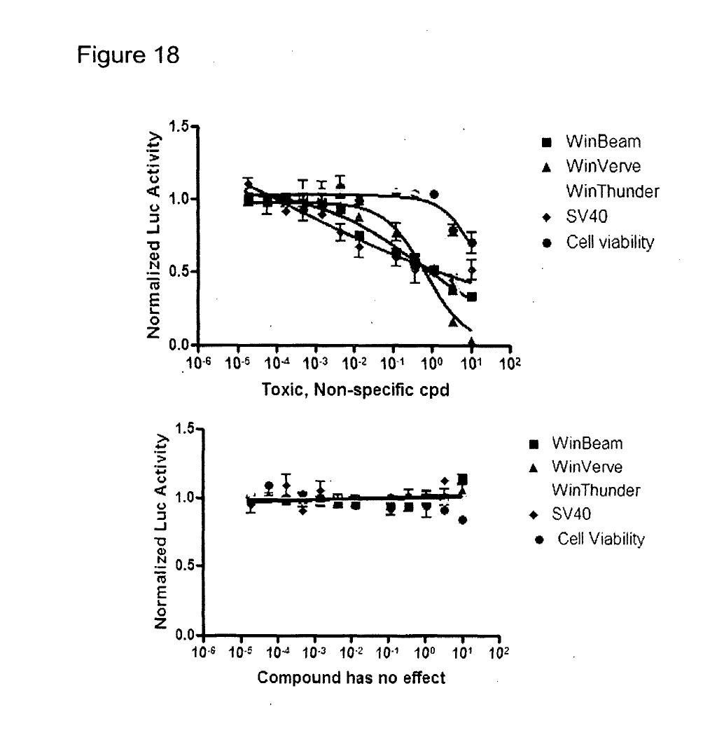

Cultured cWinBeam cells are distributed at approximately 10,000 cells per well into 384 well multiwell plates. Compounds from a compound library are added to the wells to a final concentration of 50 picomolar to 10 micomolar. A series of control wells for each cell type receive only buffer or compound solvent. Twenty four hours after the addition of compound, the activity of the WIN reporters (WinBeam, WinVerve and WinThunder) and SV40 luciferases are assayed by the addition of BrightGlo Luciferase® reporter assay reagent reaction/lysis buffers and reading light emission using a luminometer. On duplicate plates, viability of cells was assayed by the addition of CellTiter Glo reagent/lysis buffers and reading light emission using a luminometer (FIGS. 16-18). FIG. 16 shows compounds selected using WIN reporters. FIG. 17 shows how non-specific and toxic compounds are excluded from analysis using WIN reporters. FIG. 18 shows how toxic and non-specific compounds with no effect on the WIN reporters are excluded from analysis.

Test compounds were identified as candidate modulators if they modulate WinBeam reporter activity, but not affect SV40 promoter activity. In other embodiments, test compounds were considered as candidate modulators if they modulate WinVerve and WinThunder reporter activities. Test compounds which affected cell viability, as indicated by reduction in cell viability assay readings, were identified as toxic compounds (FIGS. 16-18).

Test compounds thereby identified as candidate modulators are used in repeat screenings. In the repeat screenings cWinBeam cells are distributed in wells of duplicate 96 well plates, in which a given test compound is added. The wells are assayed as described 4-48 hours after the addition of test compound. Both luciferase assays and cell viability assays were done. A test compound that results in a difference WIN luciferase signal in cWinBeam reporter cells, but not affect cell viability is identified as a Wnt signaling modulator. In other embodiments, test compounds were considered as candidate modulators if they modulate WinVerve and WinThunder reporter activities but not affect viability [FIGS. 16-18].

### Example 2

### Cell-Based Assays Using Normal Cells or Cancer Cells, Exogenous Wnt Pathway Activator Expressed by Cell

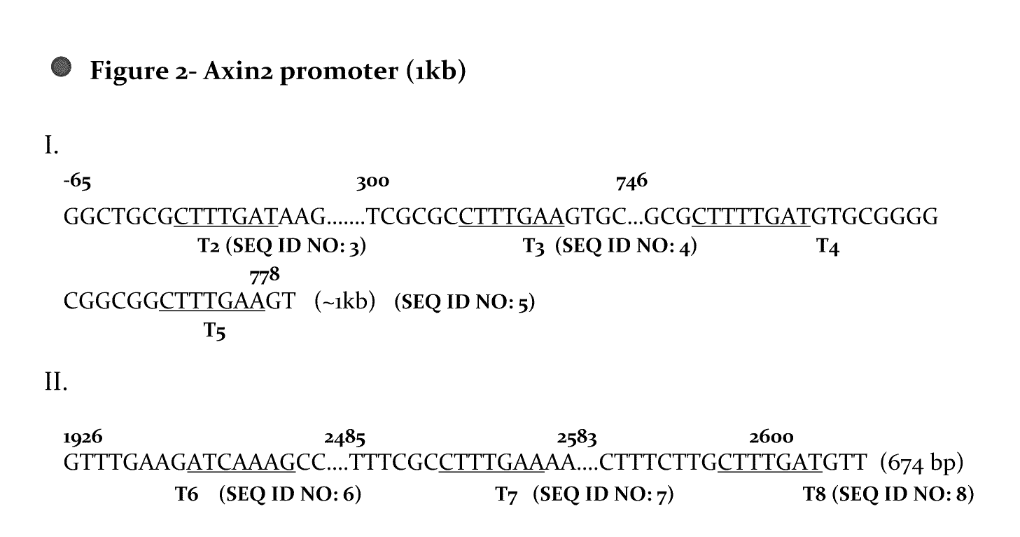

The human embryonic kidney cell line HEK293 is transiently transfected with: 1) a gene encoding β-catenin under the constitutive control of the cytomegalovirus (CMV) promoter and linked by an IRES to a gene encoding a red fluorescent protein, and 2) a reporter gene construct that includes the gene for green fluorescent protein (GFP) under the control of the axin2 promoter (FIG. 2). The RFP gene linked by an IRES to the β-catenin gene provides a marker for transfection of the cells by the Wnt activator β-catenin gene, as well as an expression control for normalization of the GFP reporter gene signal. The RFP and GFP genes encode destabilized versions of RFP and GFP having shortened half-lives for improved assays (available from Clonetech, Mountain View, Calif.).

The cells are distributed into 384 well dishes, and twenty-four hours after transfection, test compounds of a compound library are added to the wells to a final concentration of elam50 picomolar to 10 micromolar. A series of control wells receive compound buffer or solvent in place a test compound. At time points four, eight, twelve, and twenty-four hours after compound addition, the cells are assayed for RFP and GFP expression using a fluorimeter. The GFP signal of each well is normalized to the RFP signal of the well. Test compounds that increase or decrease the normalized GFP signal are identified as compounds that modulate Wnt signaling.

### Example 3

### Cell Based Assays Using Normal Cells or Cancer Cells, Exogenous Wnt Pathway Activator Inducibly Expressed by Cell, Two Reporter Genes

The normal human large intestinal epithelial cell line NCM460 (Incell Corporation, San Antonio, Tex.) is transformed with three different lentiviral constructs to have the following: 1) a stably integrated green fluorescent protein gene under the control of the WIN promoter (FIG. 1); 2) a stably integrated red fluorescent protein gene under the control of the sp5 promoter (FIG. 3); and 3) a stably integrated gene encoding Yellow Fluorescent Protein (YFP) under the control of the constitutive HSV tk promoter for gene expression normalization. The GFP, RFP, and YFP genes encode destabilized versions of the proteins for more reliable assays. The resulting NCM460/G,R,YFP-tetβcat cell line also has 4) a retrovirally integrated gene encoding a gain-of-function “E3” mutant of β-catenin linked to the inducible “Tet-On” promoter. This mutant β-catenin lacks the exon 3 domain that includes phosphorylation sites S33, S37, T41, and S45, and therefore is not targeted for destruction by the β-catenin destruction complex, leading to constitutive Wnt signaling in the cell. The “Tet-On” promoter permits titratable induction by the addition of tetracycline or the tetracycline derivative doxycycline to cultures.

The cultured NCM460/G,R,YFP-tetβcat cells are suspended and distributed at approximately 10,000 cells per well into duplicate 384 well multiwell plates. β-catenin expression is induced in the cultures in the wells of one of each of the duplicate plates by the addition of 0.5 micrograms per ml of doxycycline. Twenty-four hours after doxycycline induction, test compounds from a compound library are added to a final concentration of 1 micromolar to the wells of the duplicate plates. A series of control wells for each cell type receive only buffer or solvent. The fluorescence signals from GFP, RFP, and YFP is read 0, 4, 8, 12, and 24 hours after the addition of the compounds.

The signal from the expression of each of the GFP and RFP reporter genes is normalized to the signal from YFP for each of the wells of the duplicate plates that received test compound. These normalized values are compared to the normalized values of control wells that did not have added test compounds to provide test compound modulation values (normalized values for the degree to which each test compound altered reporter activity) for each of the test compound wells. For a given compound, the modulation value of the doxycycline-induced and noninduced NCM460/RLu,GLu-indβcat wells are then compared. Compounds that resulted in a significantly different modulation value (whether positive or negative) when added to induced cells than when added to noninduced cells are identified as compounds that modulate Wnt signaling.

### Example 4

### Negative Selection Screen Using Beta Lactamase

Cells of the DLD 1 colon cancer cell line are stably transfected with an AAV construct that includes a β-lactamase gene under the control of a WIN promoter. Expression of β-lactamase from the promoter is verified by assays using the a substrate that generates a fluorescent product, such as, for example, coumarin cephlosporin fluorescein (CCF2).

To assay for compounds that repress the WIN promoter, the cells are distributed into wells of a 384 well plate at 10,000 cells per well. Test compounds are added to the wells at a concentration of 1 micromolar. A series of control wells receive buffer or compound solvent in place of a test compound. Twenty-four hours after test compound addition, C-Dox (cephalosporin derivative of doxycycline) is added to each well at a concentration of from 0.1 to 1 micromolar. The cells are incubated for a further 24 hours prior to changing the media to a medium that does not include test compound or the prodrug.

Twenty four hours, two days, and three days, later, the wells are checked for cell viability and growth. Wells in which cells are viable are identified as wells that received a test compound that modulates Wnt signaling.

The same screen can be performed using thymidine kinase as the negatively selectable marker, in which the prodrug added to the wells after the cells have incubated in the presence of test compound is, for example, acyclovir or gangcyclovir.

1. A method for identifying a compound that modulates Wnt signaling in a cell, comprising:
(a) providing a cancerous cell that comprises a reporter gene regulated by a promoter modulated by the interaction between TCF/LEF and β-catenin;
(b) providing a noncancerous cell that comprises the reporter gene regulated by a promoter modulated by the interaction between TCF/LEF and β-catenin;
(c) contacting the cancerous cell in (a) and the noncancerous cell in (b) with a test compound; and
(d) detecting a signal from expression of the reporter gene in the cancerous cell contacted with the test compound and the signal detected from expression of the reporter gene in the cancerous cell not contacted with the test compound and detecting a signal from expression of the reporter gene in the noncancerous cell contacted with the test compound and the signal detected from expression of the reporter gene in the noncancerous cell not contacted with the test compound; and
(e) identifying the test compound as a compound that modulates Wnt signaling in cancer cells if the test compound modulates the signal from expression of the reporter gene in the cancerous cell, but does not modulate the signal from expression of the reporter gene in the non-cancerous cell.

2. The method of claim 1, wherein the cancer cells are colon cancer cells, leukemia cells, lymphoma cells, melanoma cells, breast cancer cells, prostate cancer cells, hepatocarcinoma cells, or head and neck cancer cells.

3. The method of claim 2, wherein the cells are colon cancer cells, leukemia cells, or lymphoma cells.

4. The method of claim 3, wherein the cells are leukemia cells.

5. The method of claim 4, wherein the cells are Jurkat cells or K562 cells.

6. The method of claim 3, wherein the cells are colon cancer cells.

7. The method of claim 6, wherein the cells are SW48, SW480, SW116, CaCO2, DLD1, Colo320, Colo205, LS174T, HT-29, or HT-116 cells.

8. The method of claim 1, wherein the noncancerous cells are HEK 293 cells, HeLa cells, COS-7 cells, CHO cells, or NIH/3T3 cells.

9. The method of claim 1, wherein the noncancerous cells are intestinal epithelial cells, noncancerous colon cells, noncancerous lymphocytes, noncancerous epithelial cells, noncancerous breast cells, noncancerous prostate cells, or noncancerous hepatocytes.

10. The method of claim 9, wherein the cells are intestinal epithelial cells.

11. The method of claim 10, wherein the cells are normal human large intestinal epithelial cells (NHLIEC).

12. A method for identifying a compound that modulates Wnt signaling, comprising:
(a) providing a cell that comprises a nucleic acid construct comprising a gene encoding a Wnt activator or a Wnt modulator and a reporter gene regulated by a promoter modulated by the interaction between TCF/LEF and β-catenin;
(b) contacting the cell with a test compound; and
(c) identifying as a compound that modulates Wnt signaling a test compound that has the effect of modulating the signal from expression of the reporter gene in the cell contacted with the test compound with respect to the signal from expression of the reporter gene in a control cell not contacted with the test compound; whereby the effect is not seen in cells in which the nucleic acid construct comprising the gene encoding the Wnt activator or Wnt modulator is not present.

13. A method for identifying a compound that modulates Wnt signaling, comprising:
(a) providing a cell that comprises a nucleic acid construct comprising a gene encoding a Wnt activator or a Wnt modulator under the control of an inducible promoter and further comprising a reporter gene regulated by a promoter modulated by the interaction between TCF/LEF and β-catenin;
(b) inducing expression of the Wnt activator or Wnt modulator;
(c) contacting the cell with a test compound; and
(d) identifying a test compound that has the effect of modulating the signal from expression of the reporter gene in the cell contacted with the test compound with respect to the signal from expression of the reporter gene in a control cell not contacted with the test compound; whereby the effect is not obtained in cells in which the gene encoding the Wnt activator or Wnt modulator is not induced.

14. The method of claim 13, wherein the inducible promoter is a tet-regulated promoter.

15. The method of claim 12 or 13, wherein the cells comprise a nucleic acid construct comprises a gene encoding a Wnt activator.

16. The method of claim 15, wherein the Wnt pathway activator comprises a Wnt protein, Frizzled, Disheveled, LPR5, LPR6, β-catenin, APC, axin1, or GSK3β, or an isoform, truncated form, or mutant form thereof.

17. The method of claim 16, wherein the Wnt pathway activator comprises a Wnt protein.

18. The method of claim 17, wherein the Wnt pathway activator comprises Wnt1 or Wnt3 or Wnt3a.

19. The method of claim 16, wherein the Wnt pathway activator comprises Frizzled (Fz).

20. The method of claim 19, wherein the Wnt pathway activator comprises Fz1 or Fz3 or Fz5 or Fz7.

21. The method of claim 16, wherein the Wnt pathway activator comprises β-catenin or a truncated or mutated β-catenin.

22. The method of claim 16, wherein the Wnt pathway activator comprises a truncated or mutated APC.

23. The method of claim 16, wherein the Wnt pathway activator comprises a truncated or mutated axin.

24. The method of claim 16, wherein the Wnt pathway activator comprises a truncated or mutated GSK3β.

25. The method of claim 12 or 13, wherein the cells comprise a nucleic acid molecule comprising a sequence encoding a Wnt pathway modulator.

26. The method of claim 25, wherein the Wnt pathway modulator comprises LEF1, TCF1, TCF3, TCF4, CtBP, Pygo, Groucho, CtBP, p300, or a truncated or mutant form thereof.

27. The method of claim 26, wherein the Wnt pathway modulator comprises LEF1, or an isoform, truncated form, or mutant form thereof.

28. The method of claim 26, wherein the Wnt pathway modulator comprises TCF1, TCF3, or TCF4, an isoform thereof, a truncated or mutant form thereof.

29. The method of claim 28, wherein the Wnt pathway modulator comprises TCF1, an isoform thereof, or a truncated or mutant form thereof.

30. The method of claim 29, wherein the Wnt pathway modulator comprises TCF1-E, or a truncated or mutant form thereof.

31. The method of claim 28, wherein the Wnt pathway modulator comprises TCF4, an isoform thereof, or a truncated or mutant form thereof.

32. The method of claim 31, wherein the Wnt pathway modulator comprises TCF4-E or a truncated or mutant form thereof.

33. The method of claim 12 or 13, wherein the cells comprise the nucleic acid constructs comprising the genes encoding a Wnt activator and a Wnt pathway modulator.

34. The method of claim 12 or 13, further comprising performing a cellular assay on the cells.

35. The method of claim 34, wherein the cellular assay is a cell growth assay, a cell death assay, an apoptosis assay, a migration assay, or an invasion assay.

36. The method of claims 1, 12 or 13, wherein the reporter gene is a luciferase gene, a beta galactoside gene, a beta lactamase gene, a gene encoding CAT, a gene encoding a fluorescent protein, a gene encoding alkaline phosphatase, or a gene encoding thymidine kinase.

37. The method of claim 36, wherein the reporter gene is a click beetle luciferase gene, a firefly luciferase gene, a Renilla luciferase gene, or a Gaussia luciferase gene.

38. The method of claim 36, wherein the reporter gene is a gene encoding a green fluorescent protein, a gene encoding a yellow fluorescent protein, a gene encoding a red fluorescent protein, a gene encoding an orange fluorescent protein, a gene encoding a cyan fluorescent protein or a gene encoding a blue fluorescent protein.

39. The method of claim 36, wherein the reporter gene is a gene encoding a secreted alkaline phosphatase, a secreted beta galactosidase, a secreted beta lactamase, or a secreted luciferase.

40. A method for identifying a compound that modulates Wnt signaling, comprising:
(a) providing a cell that comprises a reporter gene regulated by a promoter modulated by the interaction between TCF/LEF and β-catenin, wherein the reporter gene is negatively selectable;
(b) contacting the cell with a test compound;
(c) contacting the cell with a prodrug that is converted to an active drug by the protein encoded by the reporter gene; and
(d) identifying a test compound that permits the growth of cells in the presence of the prodrug.

41. The method of claim 40, wherein the reporter gene is a thymidine kinase gene.

42. The method of claim 41, wherein the prodrug is gangcyclovir or acyclovir.

43. The method of claim 40, wherein the reporter gene is a beta lactamase gene.

44. The method of claim 43, wherein the prodrug is a cephalosporin-containing prodrug.

45. The method of claim 44, wherein the prodrug is cephalosporin conjugated phenylenediamine mustard, doxorubicin, platinum complex, taxol, or Vinca alkaloid.

46. The method of claim 45, wherein the prodrug is cephalosporin doxorubicin or 7-(4-carboxybuanamido)-cephalosporin mustard.

47. The method of claims 1, 12, 13 or 40, wherein the promoter modulated by the interaction between TCF/LEF and β-catenin is a promoter that comprises one or more Wnt response elements (WREs) and one or more GC rich regions.

48. The method of claim 47, wherein the promoter modulated by the interaction between TCF/LEF and β-catenin is a WIN promoter.

49. The method of claims 1, 12, 13 or 40, wherein the promoter modulated by the interaction between TCF/LEF and β-catenin is a naturally-occurring promoter or a portion thereof.

50. The method of claim 49, wherein the promoter modulated by the interaction between TCF/LEF and β-catenin is an axin2, cdx, sp5, DKK4, c-myc, cyclinD1, survivin, MMP7, LEF1, or TCF1 promoter, or a portion thereof.

51. The method of any of claims 1, 12, 13 or 40, wherein the cells further comprise a second reporter gene operably linked to a second promoter modulated by the interaction between TCF/LEF and β-catenin, wherein the first promoter and the second promoter are different.

52. The method of claim 51, wherein at least one of the first promoter and the second promoter is a naturally-occurring promoter or a portion thereof.

53. The method of claim 51, wherein at least one of the first promoter and the second promoter is an axin2, cdx, sp5, DKK4, c-myc, cyclinD1, survivin, MMP7, LEF1, or TCF1 promoter, or a portion thereof.

54. The method of claim 40, wherein the cells are cancer cells.

55. The method of claim 54, wherein the cells are colon cancer cells, leukemia cells, lymphoma cells, melanoma cells, breast cancer cells, prostate cancer cells, hepatocarcinoma cells, or head and neck cancer cells.

56. The method of claim 55, wherein the cells are colon cancer cells, leukemia cells, or lymphoma cells.

57. The method of claim 55, wherein the cells are leukemia cells.

58. The method of claim 57, wherein the cells are Jurkat cells or K562 cells.

59. The method of claim 57, wherein the cells are colon cancer cells.

60. The method of claim 59, wherein the cells are SW48, SW480, SW116, CaCO2, DLD1, Colo320, Colo205, LS174T, HT-29, or HT-116 cells.

61. The method of claim 40, wherein the cells are noncancerous cells.

62. The method of claim 60, wherein the cells are HEK 293 cells, COS cells, CHO cells, 3T3 cells.

63. The method of claim 61, wherein the cells are noncancerous intestinal epithelial cells, noncancerous colon cells, noncancerous lymphocytes, noncancerous epithelial cells, noncancerous breast cells, noncancerous prostate cells, or noncancerous hepatocytes.

64. The method of claim 63, wherein the cells are noncancerous intestinal epithelial cells.

65. The method of claim 64, wherein the cells are normal human large intestinal epithelial cells (NHLIEC).

66. A compound identified by a method according to claims 1, 12, 13 or 40.


SEQUENCE LISTING (15 sequences)

SEQ ID NO: 1 - DNA, 51 bp, Artificial Sequence
  Synthetic oligonucleotide
gatccctcctttgatcttaccccctttgatcttaccccctttgatcttacg

SEQ ID NO: 2 - DNA, 51 bp, Artificial Sequence
  Synthetic oligonucleotide
gatcccctctttgttcccggcctctttgttcccggcctctttgttcccggg

SEQ ID NO: 3 - DNA, 17 bp, Artificial Sequence
  Synthetic oligonucleotide
ggctgcgctttgataag

SEQ ID NO: 4 - DNA, 17 bp, Artificial Sequence
  Synthetic oligonucleotide
tcgcgcctttgaagtgc

SEQ ID NO: 5 - DNA, 34 bp, Artificial Sequence
  Synthetic oligonucleotide
gcgcttttgatgtgcggggcggcggctttgaagt

SEQ ID NO: 6 - DNA, 17 bp, Artificial Sequence
  Synthetic oligonucleotide
gtttgaagatcaaagcc

SEQ ID NO: 7 - DNA, 15 bp, Artificial Sequence
  Synthetic oligonucleotide
tttcgcctttgaaaa

SEQ ID NO: 8 - DNA, 18 bp, Artificial Sequence
  Synthetic oligonucleotide
ctttcttgctttgatgtt

SEQ ID NO: 9 - DNA, 376 bp, Mus musculus
gagataacaaagacactttgtcactttgagcactgccttggtgcaaatctttaattgcaagtgggggtgggggatcgagtgtcccgagcaacccaagcgcgagtctccaggcggcaaggccccctttgatcaggaaaatccaattatttgtgtatatacatttcccacatagtgattacttcattaattgtcccgcctttatctcctcccttcccatcccccctaataatcagttcttttatccagaccaacaaacacaccataggagctttgtggattcaaaggatttgctttcgcttctgaaagagccgctattctttgatgattgggtagcggcaaacttcaaagccataaatctttcctctgactggctggc

SEQ ID NO: 10 - DNA, 403 bp, Homo sapiens
gagataacaaagacactttgtcactttgggcgctgcgtcggtgcaaatctttaattgcaagtgtgtgtgtgtgtgtgtgtgtgtatgttggggggggcgggaagagtgtctcgagcaacccaagcgcgggtctccaggcggcaaggccccctttgatcaggaaaatccaattatttgtgtatatacatttcccacatagtgattacttcattaattgtcccgcctttatctcctcccttcccatcccccctaataatcagttcttttatccagaccaacaaacacaccataggagctttgtggattcaaaggatttgctttcgcttctgaaagagccgctattctttgatgattgggtagcggcaaacttcaaagccataaatcttccctctgactggctggc

SEQ ID NO: 11 - DNA, 403 bp, Gallus gallus
gggctgccccacaaccccttaattgcgaaaaacaaaaaaaacaaaaacaaaaaagaaaaaaacagaaaaaaacagaaaaagcctgacgggggggggggggatccggtgtatcgcgcatcccgcgagagggtctccaggcagtacggggtcctttgatcaagaaaatccaattatttgtgtatatacatttcccacatagtgattacttcattaattgtcccgcctttatctcctcccttcccatcccccctaataatcagttcttttatcccgaccaacaaacacaccataggagctttgtggattcaaaggatttgctttcgcttctgaaagagccgctattctttgatgattgggtagcggcaaacttcaaagccataaatcttccttctgactggctggc

SEQ ID NO: 12 - DNA, 399 bp, Danio rerio
gacatatacatataagcctgtcggagatctctggagataacacacacagtcgcattgtcgcacaacaaatccttaattacagaaagagggatcgggtgactccggcatccaatagcgggggtctccaggcgggcgaagggggtctttgatcaagaaaatccaattatttgtgtatatacatttcccacatagtgattacttcattaattgtcccgcctttatccccacccgtcccatccccccctaataatcagcccttttatccagaccaacaaacacaccataggagctttgtgtggattcagaggatttgcttccccctctcaaagagccgctcttctttgatgattgggcagcggcgaacttcaaagtcataaatctcactcctgactggctggc

SEQ ID NO: 13 - DNA, 396 bp, Xenopus tropicalis
gaggaaagcaattcccttcctctctgggcaaataacaaaggcatattgtcactttgctacaaatccttcattacacagaggaagggggaatccaatgctcagagctctggaccagggggtctccgggagctcaggggtctctttgatcaggaaaatccaattatttgtgtatatacattccccacatagtgattacttcattaattgtcccgccttttgtcacctccctttccacccctgtgtaataatcagttcctttatgtcgccccggccaacactccatgagggctttgtacattcaaaggatttgcttgggcttctgaaagagccgctgttctttgatgattgggcagcggcaaacttcaaaggcagagaggcgggctgtgagtggctggc

SEQ ID NO: 14 - DNA, 416 bp, Homo sapiens
ctcgagggcatcctctatagcatattggccagtatgattcatcctttctttgagcacaattagctgtgaaaacagatcttagggcagactccctccctacccccaaaggattactgaaggtaggaaatgcaggtgattatcagagtttgcctttgatacagacatcctgctctgccgtggccctttgaactaactttgatatgcaataattaagagggattgaacccctggaggagaagccgcatggccctgcctcttctctccttctattgagtccttgttttgaactattgatcaaacagatttgaagggatttgttgaagcctgtgtggggcaggaggaaggaacggagcggggaggaggcacatctggttataaaagcttggcattccggtactgttggtaaagccaccatg

SEQ ID NO: 15 - DNA, 108 bp, Artificial Sequence
  Synthetic oligonucleotide
cctttgatccctttgatccctttgatccctttgatccctttgatccctttgatccctttgatccctttgatccctttgatccctttgatccctttgatccctttgatc